(19) United States
(12) Patent Application Publication
(10) Pub. No.: US 2025/0344935 A1
(43) Pub. Date: Nov. 13, 2025

(54) MEDICAL SYSTEM, ENDOSCOPE CONTROL DEVICE, ENDOSCOPE CONTROL METHOD, AND STORAGE MEDIUM

(71) Applicant: OLYMPUS MEDICAL SYSTEMS CORP., Tokyo (JP)

(72) Inventor: Noriaki YAMANAKA, Tokyo (JP)

(73) Assignee: OLYMPUS MEDICAL SYSTEMS CORP., Tokyo (JP)

(21) Appl. No.: 19/203,399

(22) Filed: May 9, 2025

Related U.S. Application Data
(60) Provisional application No. 63/645,385, filed on May 10, 2024.

(51) Int. Cl. A61B 1/00; A61B 1/018

(52) U.S. Cl. CPC A61B 1/00006; A61B 1/00114; A61B 1/00133; A61B 1/018

(57) ABSTRACT

The present disclosure provides an endoscope control device comprising: at least one processor comprising hardware, the at least one processor being configured to: control an actuator connected to at least one wire of an endoscope to drive the at least one wire and bend a bending section at a distal end of the endoscope to a first angle, and when a bend angle of the bending section is returned to a second angle smaller than the first angle by loosening the at least one wire from the state where the bending section is bent to the first angle, control the actuator to drive the at least one wire to maintain the bend angle at the second angle.

### CROSS-REFERENCE TO RELATED APPLICATIONS

This application is based upon and claims the benefit of priority from U.S. Provisional Application No. 63/645,385, filed on May 10, 2024; the entire contents of which are incorporated herein by reference.

### TECHNICAL FIELD

The present disclosure relates to medical systems, endoscope control devices, endoscope control methods, and storage media.

### BACKGROUND ART

A known electric endoscope in the related art bends the distal end of an insertion section by driving a wire (e.g., see Patent Literature 1). This endoscope is equipped with a position sensor at the distal end. The endoscope uses the position sensor to detect positional displacement at the distal end caused by an insertion load of a treatment tool inserted into a channel, and controls the tension of the wire to return the distal end to the position prior to the occurrence of the positional displacement.

### CITATION LIST

### Patent Literature

{PTL 1}

### The Publication of US Patent Application No. 2019/000568

### SUMMARY

An aspect of the present disclosure provides an endoscope control device comprising: at least one processor comprising hardware, the at least one processor being configured to: control an actuator connected to at least one wire of an endoscope to drive the at least one wire and bend a bending section at a distal end of the endoscope to a first angle, and when a bend angle of the bending section is returned to a second angle smaller than the first angle by loosening the at least one wire from the state where the bending section is bent to the first angle, control the actuator to drive the at least one wire to maintain the bend angle at the second angle.

Another aspect of the present disclosure provides an endoscope control method comprising: controlling an actuator connected to at least one wire of an endoscope to drive the at least one wire and bend a bending section at a distal end of the endoscope to a first angle, and when a bend angle of the bending section is returned to a second angle smaller than a first angle by loosening the at least one wire from a state where the bending section is bent to the first angle, using at least one processor to control the actuator to drive the at least one wire to maintain the bend angle at the second angle.

Another aspect of the present disclosure provides a computer-readable non-transitory storage medium that stores an endoscope control program causing a computer to execute the aforementioned endoscope control method.

Another aspect of the present disclosure provides a medical system including: an endoscope including a bending section, which is disposed at a distal end of an insertion section to be inserted into a body, and at least one wire connected to the bending section; a driving device that is connected to the endoscope and that drives the wire; and an endoscope control device that controls the driving device. The endoscope control device includes at least one processor. When a bend angle of the bending section is returned to a second angle smaller than a first angle by loosening the wire from a state where the bending section is bent to the first angle by pulling the wire, the processor is configured to control the driving device to execute bend maintaining control for pulling the wire within a range in which the bend angle is maintained at the second angle.

### BRIEF DESCRIPTION OF DRAWINGS

FIG. 1 is an overall configuration diagram illustrating the configuration of a medical system according to a first embodiment.

FIG. 2 illustrates an insertion section of an endoscope of the medical system in FIG. 1.

FIG. 3 is a cross-sectional view illustrating the insertion section of the endoscope of the medical system in FIG. 1.

FIG. 4 is a vertical sectional view illustrating a coupling section of the endoscope of an endoscope system in FIG. 1.

FIG. 5A illustrates a wire attachable-detachable section, prior to being attached to the driving device, and a wire driver in the endoscope system in FIG. 1.

FIG. 5B illustrates the wire attachable-detachable section, attached to the driving device, and the wire driver in the endoscope system in FIG. 1.

FIG. 6 illustrates a treatment-tool sensor disposed radially outward of a transparent section provided in a part of the channel tube disposed in the coupling section.

FIG. 7 is a block diagram illustrating an endoscope control device according to this embodiment.

FIG. 8 illustrates the relationship among the drive amount, the tension, and the bend angle of a wire.

FIG. 9A illustrates a state where a bending section of the insertion section in FIG. 2 is not pulled.

FIG. 9B illustrates a state where the bending section in FIG. 9A is pulled to cause the bend angle of the bending section to become the first angle.

FIG. 9C illustrates a state where the bending section in FIG. 9B is loosened to cause the bend angle of the bending section to transition from the first angle to a second angle.

FIG. 9D illustrates a state where the bending section in FIG. 9C is loosened to cause the bending section to reach the second angle.

FIG. 9E illustrates a state where the bending section in FIG. 9D is pulled to cause the bending section to be maintained at the second angle.

FIG. 10 is a flowchart illustrating an endoscope control method executed by the endoscope control device in FIG. 7.

### DESCRIPTION OF EMBODIMENTS

A medical system 100, an endoscope control device 40, an endoscope control method, and a storage medium according to an embodiment of the present disclosure will now be described with reference to the drawings.

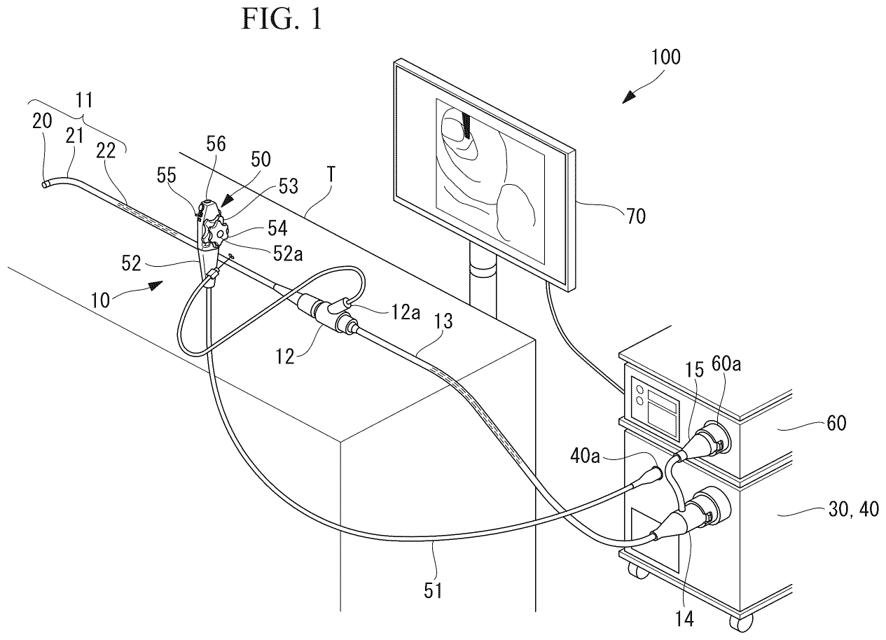

As shown in FIG. 1, the medical system 100 according to this embodiment is a system for observing and treating the inside of the body of a patient lying on a surgical table T. The medical system 100 includes an endoscope 10, a driving device 30, and the endoscope control device 40. In this embodiment, the driving device 30 and the endoscope control device 40 are accommodated within the same cabinet. The medical system 100 further includes a manipulation device 50, a video control device 60, and a display device 70.

The endoscope 10 is a flexible endoscope to be inserted into the patient's lumen. An endoscopic image acquired by the endoscope 10 is input to the display device 70 via the video control device 60, so as to be displayed on the display device 70.

The manipulation device 50 is connected to an adapter 40a of the endoscope control device 40 via a manipulation cable 51. An operation input received by the manipulation device 50 is input to the endoscope control device 40 from the manipulation device 50. The endoscope control device 40 controls the driving device 30 based on the operation input received by the manipulation device 50. Accordingly, the endoscope 10 is operated in accordance with the operation input.

The endoscope 10 is detachably connected to the driving device 30. In the description hereinafter, the side of the endoscope 10 to be inserted into the patient's lumen will be referred to as the distal end, whereas the side to be attached to the driving device 30 will be referred to as the proximal end.

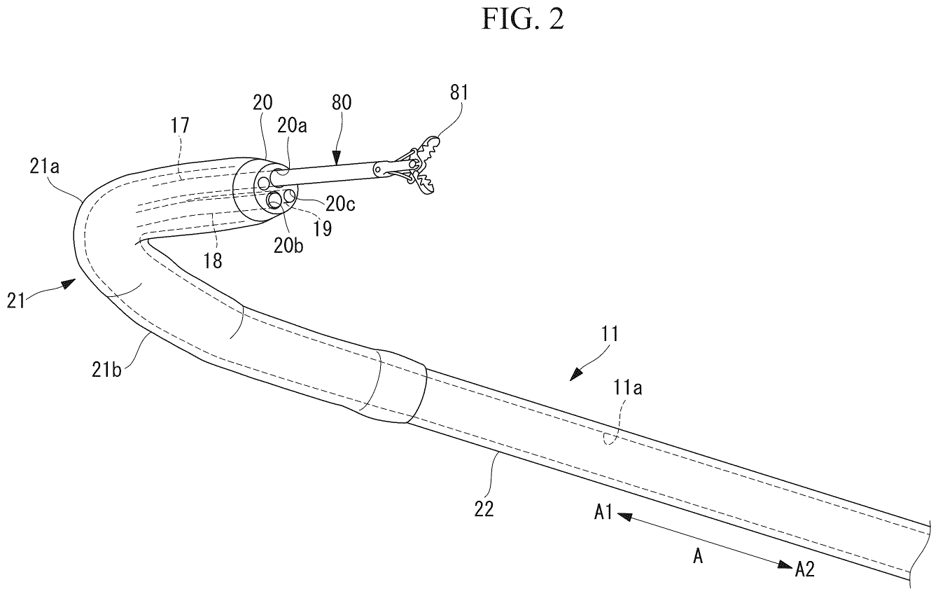

As shown in FIG. 1, the endoscope 10 includes an insertion section 11, a coupling section 12, an external flexible section 13, a first attachable-detachable section 14, and a second attachable-detachable section 15, in that order from the distal end. The insertion section 11 is a long flexible member. As shown in FIG. 2, the insertion section 11 has an internal path 11a therein.

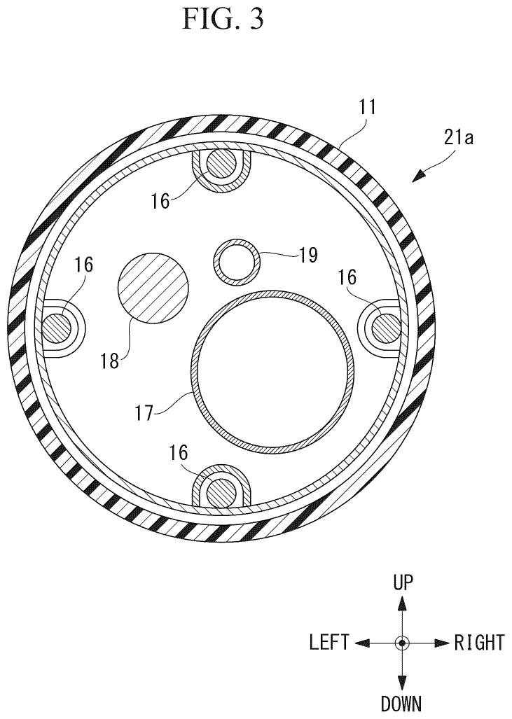

The internal path 11a of the endoscope 10 extends in a longitudinal direction A of the endoscope 10 from the distal end of the insertion section 11 to the proximal end of the first attachable-detachable section 14. As shown in FIG. 3, the internal path 11a accommodates, for example, wires 16, a channel tube 17, a light guide 18, and an imaging cable 19 that are to be described later.

The insertion section 11 has a distal end section 20, a bending section 21, and an internal flexible section 22, in that order from the distal end.

As shown in FIG. 2, the distal end section 20 includes an opening 20a communicating with the channel tube 17, an illuminating unit 20b, and an imaging unit 20c. A treatment unit 81, such as grasping forceps, provided at the distal end of a treatment tool 80 extending through a channel within the channel tube 17 protrudes and retracts through the opening 20a. The light guide 18 is connected to the illuminating unit 20b, and the imaging cable 19 is connected to the imaging unit 20c.

The bending section 21 has a first bending section 21a and a second bending section 21b provided at the proximal end of the first bending section 21a. Each of the first bending section 21a and the second bending section 21b is bendable upward, downward, leftward, and rightward.

As shown in FIG. 3, the first bending section 21a is connected to four wires 16 for bending the first bending section 21a upward, downward, leftward, and rightward, respectively. Similarly, the second bending section 21b is also connected to other four wires 16 for bending the second bending section 21b upward, downward, leftward, and rightward, respectively. The first bending section 21a and the second bending section 21b are independently bendable in different directions.

The coupling section 12 couples the internal flexible section 22 of the insertion section 11 and the external flexible section 13 to each other. The coupling section 12 is provided with an insertion port 12a for inserting the treatment tool 80 into the channel tube 17 in the internal path 11a.

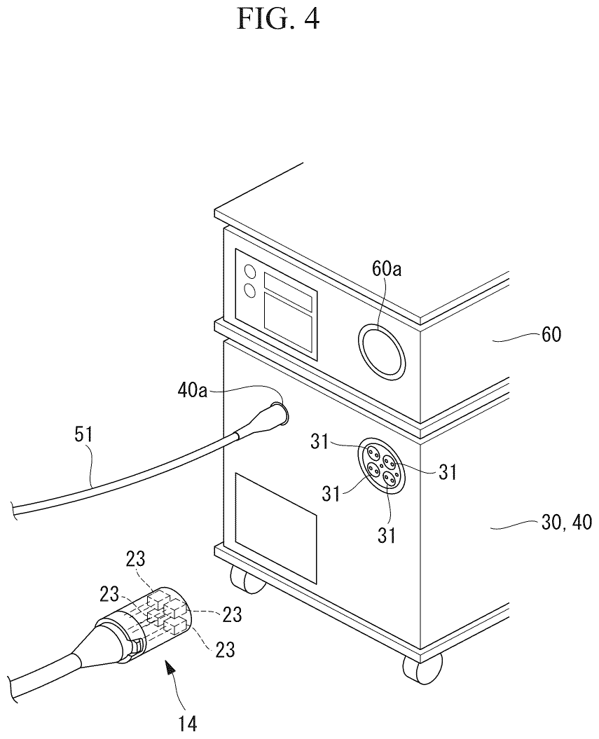

As shown in FIG. 4, the first attachable-detachable section 14 has four wire attachable-detachable sections 23 as a mechanism for detachably connecting the wires 16 to the driving device 30. Each wire attachable-detachable section 23 is provided at the proximal end of a pair of wires 16 and attaches and detaches the wires 16 to and from the driving device 30. For example, the four wire attachable-detachable sections 23 respectively attach and detach a pair of wires 16 for bending the first bending section 21a upward and downward, a pair of wires 16 for bending the first bending section 21a leftward and rightward, a pair of wires 16 for bending the second bending section 21b upward and downward, and a pair of wires 16 for bending the second bending section 21b leftward and rightward to and from the driving device 30.

The second attachable-detachable section 15 is detachably connected to an adapter 60a of the video control device 60. The light guide 18 and the imaging cable 19 are connected to the video control device 60 via the second attachable-detachable section 15.

The driving device 30 is connected to a power source (not shown) and operates in accordance with electric power supplied from the power source. The driving device 30 has four wire drivers 31 as a mechanism for driving the wires 16. By being connected to the first attachable-detachable section 14, the four wire drivers 31 are respectively coupled to the four wire attachable-detachable sections 23 and can each drive the corresponding pair of wires 16. For example, the four wire drivers 31 respectively drive the pair of wires 16 for bending the first bending section 21a upward and downward, the pair of wires 16 for bending the first bending section 21a leftward and rightward, the pair of wires 16 for bending the second bending section 21b upward and downward, and the pair of wires 16 for bending the second bending section 21b leftward and rightward.

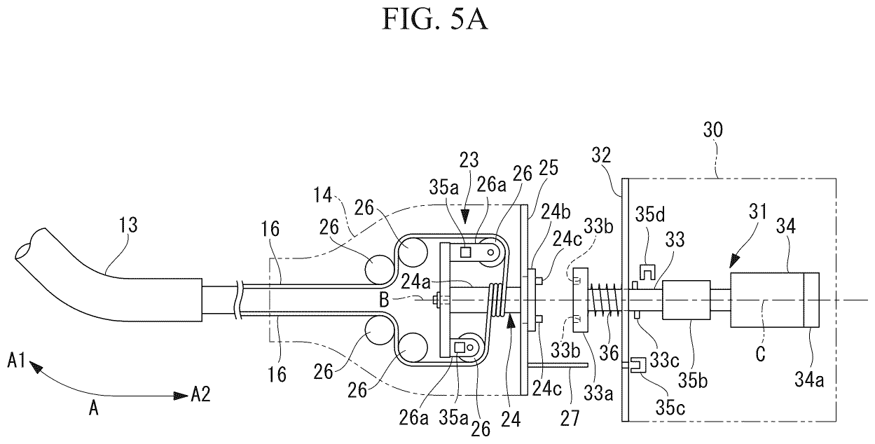

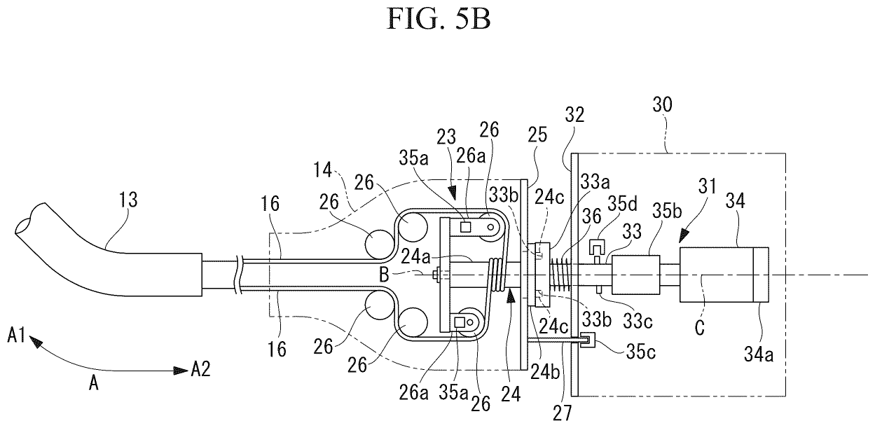

FIGS. 5A and 5B illustrate the configuration of each wire attachable-detachable section 23 and each wire driver 31. FIG. 5A illustrates the wire attachable-detachable section 23 and the wire driver 31 in a state where they are separated from each other, and FIG. 5B illustrates the wire attachable-detachable section 23 and the wire driver 31 in a state where they are connected to each other.

For example, FIGS. 5A and 5B illustrate the wire attachable-detachable section 23, including the pair of wires 16 for bending the first bending section 21a upward and downward, and the wire driver 31. Since the remaining wire attachable-detachable sections 23 and the remaining wire drivers 31 also have the configuration in FIGS. 5A and 5B, redundant descriptions will be omitted.

Each wire attachable-detachable section 23 has a rotating drum 24 and a support member 25 for supporting the rotating drum 24. The support member 25 is to be secured to a support member 32 of the wire driver 31 in a state where the wire attachable-detachable section 23 is coupled to the wire driver 31.

The rotating drum 24 is supported by the support member 25 in such a manner as to be rotatable around a rotation axis B extending in the longitudinal direction A of the insertion section 11. The rotating drum 24 includes a winding pulley 24a disposed coaxially with the rotation axis B and a coupling 24b fixed to the winding pulley 24a.

The pair of wires 16 are guided to the winding pulley 24a via at least one pulley 26 that guides the wires 16, and are wound around the outer peripheral surface of the winding pulley 24a. The winding pulley 24a rotates around the rotation axis B so as to pull or feed the pair of wires 16.

The coupling 24b is a disk member that is fixed to the proximal end of the winding pulley 24a and that is disposed coaxially with the rotation axis B, and is exposed at the proximal end of the wire attachable-detachable section 23. The surface at the proximal end of the coupling 24b is provided with two engagement protrusions 24c at opposite sides of the rotation axis B.

Each wire attachable-detachable section 23 includes a dog 27 that is provided on the support member 25 and that is for detecting whether the wire attachable-detachable section 23 is attached to or detached from the wire driver 31.

The dog 27 is a member protruding outward of the wire attachable-detachable section 23 from the support member 25 and exposed at the proximal end of the wire attachable-detachable section 23, and is, for example, a pin-shaped member extending parallel to the rotation axis B. As shown in FIG. 5B, in a state where the wire attachable-detachable section 23 is connected to the wire driver 31, the dog 27 extends through the support member 32 of the wire driver 31 and is inserted into the wire driver 31.

The wire driver 31 has a shaft 33, an actuator, such as a motor 34, connected to the shaft 33, and the support member 32 that rotatably supports the shaft 33.

The shaft 33 is supported by the support member 32 in such a manner as to be rotatable around a rotation axis C and extendable and retractable in the longitudinal direction A. The rotation axis C is the center axis of the shaft 33 and is aligned with the rotation axis B of the rotating drum 24 in a state where the first attachable-detachable section 14 is connected to the driving device 30.

The motor 34 is, for example, a direct-current motor. The motor 34 generates a rotational force as a driving force in accordance with the electric power supplied from the power source, and rotates the shaft 33 around the rotation axis C. In the wire driver 31, the motor 34 is provided with an encoder 34a that detects the rotational speed and the rotational angle of the motor 34. The encoder 34a is connected to the proximal end of the motor 34.

The wire driver 31 has a coupling 33a as a mechanism that is provided at the shaft 33 and that couples the motor 34 to the rotating drum 24.

The coupling 33a is a disk member that is fixed to the distal end of the shaft 33 and that is disposed coaxially with the rotation axis C, and rotates integrally with the shaft 33. The coupling 33a is exposed at the distal end of the wire driver 31. The surface at the distal end of the coupling 33a is provided with two engagement recesses 33b at opposite sides of the rotation axis C.

As shown in FIG. 5B, with the engagement between the engagement protrusions 24c and the engagement recesses 33b, the coupling 24b and the coupling 33a become coupled to each other, whereby the motor 34 becomes coupled to the wires 16 via the rotating drum 24. In this state, the rotating drum 24, the coupling 24b, the coupling 33a, and the shaft 33 are integrally rotatable around the rotation axes B and C. Therefore, the rotational force (driving force) generated by the motor 34 is transmitted as a force acting in the longitudinal direction A to the wires 16 via the rotating drum 24.

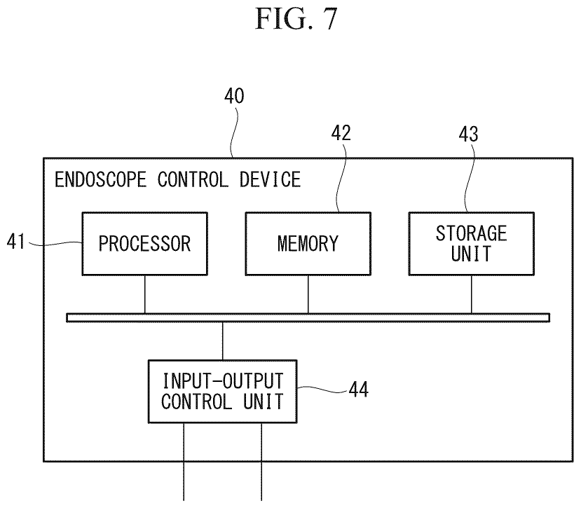

When the motor 34 is controlled by the processor (41-see FIG. 7) to rotate in one direction around the rotation axis C, the rotating drum 24 is rotated in one direction around the rotation axis B. As a result, for example, one of the wires 16 of the pair disposed at opposite sides in the up-down direction is pulled, whereas the other is loosened. In contrast, when the motor 34 is rotationally driven in the other direction, the wires 16 that are pulled and loosened are interchanged. Thus, the rotational driving direction of the motor 34 is switched, so that the bending section 21 can be bent either upward or downward. The same applies to the leftward and rightward directions.

The medical system 100 further includes a tension sensor (first sensor) 35a, a torque sensor 35b, an attachment-detachment sensor 35c, a coupling sensor 35d, an electric-current sensor (not shown), and a treatment-tool sensor (second sensor) 35e.

The tension sensor 35a is provided for each of the four wire attachable-detachable sections 23. The torque sensor 35b, the attachment-detachment sensor 35c, the coupling sensor 35d, and the electric-current sensor are provided for each of the four wire drivers 31. The treatment-tool sensor 35e is provided at, for example, the coupling section 12. The sensors 35a, 35b, 35c, 35d and 35e are connected to the endoscope control device 40. An output from each of the sensors 35a, 35b, 35c, 35d, and 35e is successively transmitted to the endoscope control device 40.

The tension sensor 35a is provided for each wire 16. For example, the tension sensor 35a is constituted of a distortion sensor installed in a support pillar 26a of the pulley 26 and detects the tension of the wire 16 in accordance with distortion of the support pillar 26a. By using the tension sensor 35a to detect the tension for every drive amount of the wire 16, information related to a change in the tension of the wire 16 can be acquired.

The torque sensor 35b is provided for each motor 34 and detects the torque of the motor 34. For example, the torque sensor 35b is attached to the shaft 33 and detects torque acting around the rotation axis C as the torque of the motor 34.

The attachment-detachment sensor 35c detects whether the wire attachable-detachable section 23 is attached to or detached from the wire driver 31. When the wire attachable-detachable section 23 is connected to the wire driver 31, the attachment-detachment sensor 35c engages with the dog 27 extending through the support member 32 and inserted into the wire driver 31. The attachment-detachment sensor 35c has, for example, an optical sensor that detects that the dog 27 is in contact therewith or is in close vicinity thereof, and uses the optical sensor to detect engagement with the dog 27.

The coupling sensor 35d is provided for each motor 34. The coupling sensor 35d detects engagement between the coupling 24b and the coupling 33a based on shifting of the shaft 33, so as to detect that the motor 34 is coupled to the wires 16.

As shown in FIG. 5B, the coupling 33a is pressed by the coupling 24b, so as to move toward a proximal end A2 together with the shaft 33. The coupling sensor 35d has, for example, an optical sensor that detects that a dog 33c provided on the shaft 33 is approaching, and detects engagement between the coupling 24b and the coupling 33a based on the approaching of the dog 33c.

The coupling 33a is biased toward a distal end Al by an elastic member 36, such as a compression spring, disposed between the coupling 33a and the support member 32. As shown in FIG. 5A, in a state where the wire attachable-detachable section 23 and the wire driver 31 are separated from each other, the coupling 33a moves toward the distal end A1 together with the shaft 33 in accordance with the biasing force of the elastic member 36, so that the dog 33c is disposed at a position located away from the coupling sensor 35d. In this state, the coupling sensor 35d does not detect engagement between the coupling 24b and the coupling 33a.

The electric-current sensor is provided for each motor 34, and detects an electric current flowing to the motor 34.

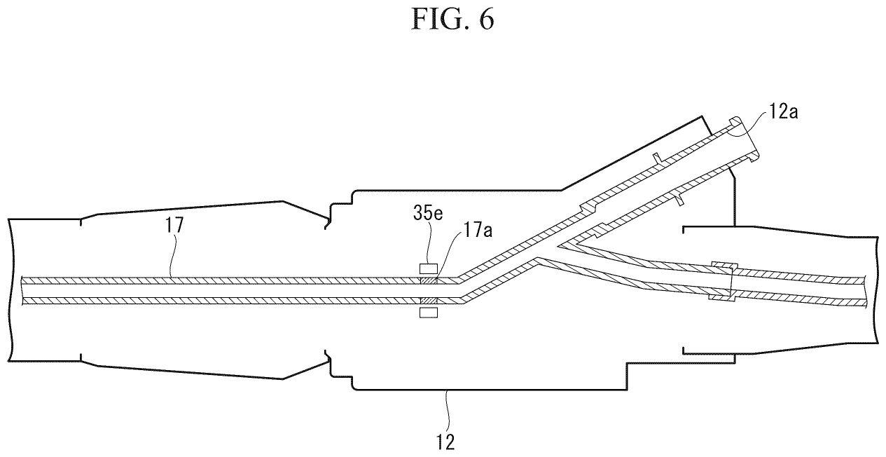

As shown in FIG. 6, for example, the treatment-tool sensor 35e is disposed radially outward of a transparent section 17a provided in a part of the channel tube 17 disposed in the coupling section 12, and is for detecting the treatment tool 80 extending through the interior. The treatment-tool sensor 35e is, for example, an optical sensor. The treatment-tool sensor 35e may be disposed at the proximal end, the distal end, or any position in the longitudinal direction of the channel tube 17.

The endoscope control device 40 acquires an operation input from the manipulation device 50 via the adapter 40a. The endoscope control device 40 controls the driving device 30 based on the acquired operation input.

As shown in FIG. 7, the endoscope control device 40 is a program-executable computer that includes at least one processor 41, at least one memory 42, a storage unit 43 capable of storing a program and data, and an input-output control unit (interface) 44.

The storage unit 43 is a non-transitory nonvolatile recording medium that stores programs and required data, and is, for example, a ROM or a hard disk. The functions, to be described later, of the endoscope control device 40 are implemented as a result of an endoscope control program stored in the storage unit 43 being loaded to the memory 42 and being executed by the processor 41. At least one or more of the functions of the endoscope control device 40 may be implemented by a dedicated logical circuit.

The manipulation device 50 is used by an operator, such as a surgeon, for inputting an operation for driving the endoscope 10. The received operation input is transmitted to the endoscope control device 40 via the manipulation cable 51.

As shown in FIG. 1, the manipulation device 50 includes a main body 52, a first angle knob 53, and a second angle knob 54. The manipulation device 50 also includes a switch 55 and an audio sensor 56.

The main body 52 has, for example, a shape that can be held with the left hand of the operator, such as a surgeon.

The first angle knob 53 and the second angle knob 54 are attached to the main body 52 in such a manner as to rotate around the same rotation axis 52a.

For example, when the operator uses the right hand to rotate the first angle knob 53, the wires 16 for bending the bending section 21 upward and downward are driven. For example, when the second angle knob 54 is rotated, the wires 16 for bending the bending section 21 leftward and rightward are driven.

The input-output control unit 44 is connected to the driving device 30, the manipulation device 50, and the display device 70. Based on control of the processor 41, the input-output control unit 44 performs exchanging of data and exchanging of control signals with connected devices.

Next, the endoscope control method executed by the endoscope control device 40 according to this embodiment will be described below.

In the endoscope control device 40 according to this embodiment, the processor 41 acquires an operation input generated as a result of the operator, such as a surgeon, operating the first angle knob 53 or the second angle knob 54 of the manipulation device 50, and controls the driving device 30.

In detail, the processor 41 actuates any of the motors 34 corresponding to the operation input in the driving device 30, thereby pulling and loosening the wires 16. Accordingly, the bending section 21 can be bent in a desired direction by a desired bend angle. As compared with a state where the bending section 21 is straight, the bend angle is an angle changed when the bending section 21 is bent, assuming that the straight state is 0 degrees. Alternatively, it is possible to determine this from an imaging direction at the distal end of the bending section 21. Assuming that the imaging direction when the bending section 21 is straight is 0 degrees, the aforementioned angle is an angle between the imaging direction when the bending section 21 is straight and the imaging direction after the bending section 21 is bent.

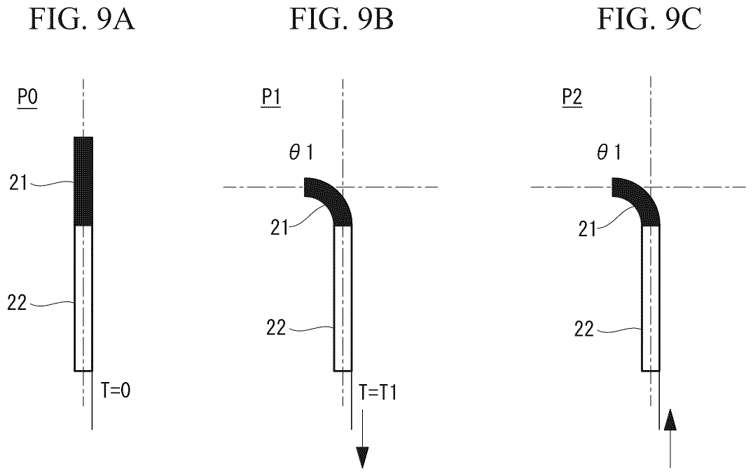

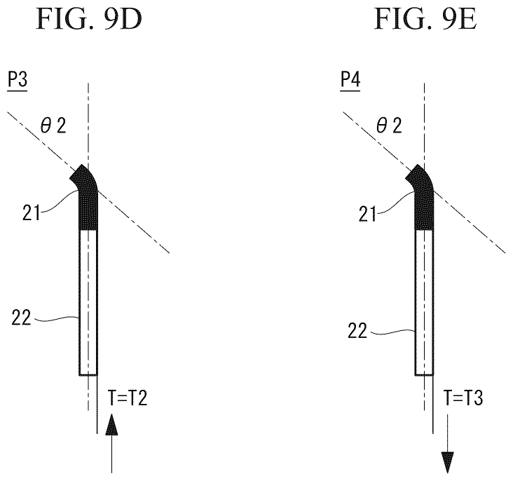

As shown in FIGS. 9A and 9B, the processor 41 controls the driving device 30 to pull one of the wires 16 such that the bending section 21 bends in an arbitrary direction. Furthermore, as shown in FIG. 9C, the processor 41 controls the driving device 30 to reach the state of P2 where the wire 16 is loosened from the state of P1 where the bending section 21 is bent to an arbitrary first angle θ1 in an arbitrary direction by pulling the wire 16. Moreover, as shown in FIG. 9D, when the state of P3 is reached as a result of further loosening the wire 16 to decrease the bend angle of the bending section 21 to a second angle θ2 smaller than the first angle θ1, the processor 41 controls the driving device 30 to execute bend maintaining control. The bend maintaining control is the state of P4 shown in FIG. 9E and involves pulling the loosened wire 16 within a range in which the bend angle is maintained at the second angle θ2. The first angle θ1 and the second angle θ2 are arbitrary.

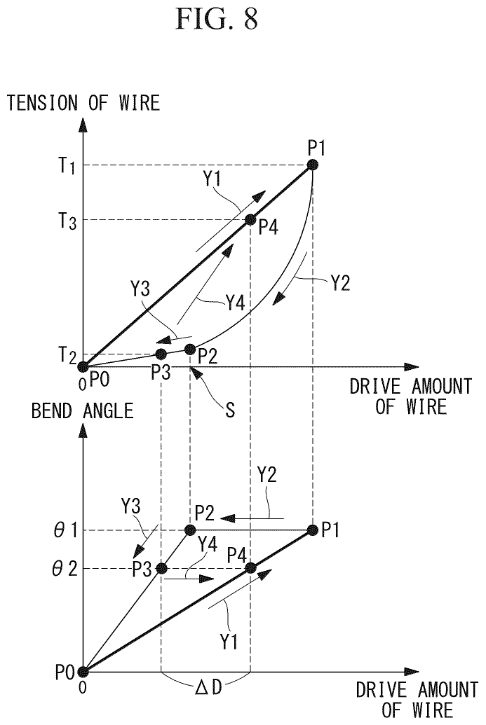

FIG. 8 illustrates the relationship between the drive amount of the wire 16 and the tension of the wire 16, as well as the relationship between the drive amount of the wire 16 and the bend angle of the bending section 21. For example, starting from an origin point where the drive amount of the wire 16 is zero, when the wire 16 is pulled, the tension and the bend angle of the wire 16 increase substantially linearly in proportion to the drive amount of the wire 16, as indicated by an arrow Y1.

However, after the bend angle of the bending section 21 reaches the arbitrary first angle θ1, when the bend angle is to be decreased by loosening the wire 16, the tension of the wire 16 decreases rapidly without being proportional to the drive amount of the wire 16 until the drive amount of the wire 16 decreases to a predetermined point S, as indicated by an arrow Y2.

Specifically, the operator first rotates the first angle knob 53 or the second angle knob 54 in one direction, so as to cause the drive amount of one of the wires 16 to increase and to cause the bend angle of the bending section 21 to reach the first angle θ1. Subsequently, when the operator rotates the first angle knob 53 or the second angle knob 54 in the other direction to decrease the drive amount of the wire 16, the tension of the wire 16 greatly decreases without causing the bend angle to change.

Then, when the drive amount of the wire 16 decreases to the predetermined point S, the bend angle starts to decrease together with the decrease in the drive amount of the wire 16 at that point, as indicated by an arrow Y3. Then, the tension and the bend angle of the wire 16 start to decrease substantially linearly again in proportion to the drive amount of the wire 16. Thus, the relationship between the drive amount and the tension of the wire 16 and the relationship between the drive amount and the bend angle of the wire 16 each form a hysteresis curve in which the path differs between when the wire 16 is pulled and when the wire 16 is loosened.

Then, after the bend angle becomes the second angle θ2 as a result of the drive amount of the wire 16 decreasing to T2 that is smaller than the tension at the predetermined point S, the drive amount of the wire 16 is increased again to T3 that is larger than T2. This causes the tension of the wire 16 to increase without causing the bend angle to change, as indicated by an arrow Y4.

In this embodiment, when the second angle θ2 is reached from the first angle θ1 that is larger than the second angle θ2, the processor 41 controls the driving device 30 to perform the bend maintaining control. Accordingly, as indicated by the arrow Y4 in FIG. 8, the tension of the wire 16 can be increased within a range in which the bend angle does not change.

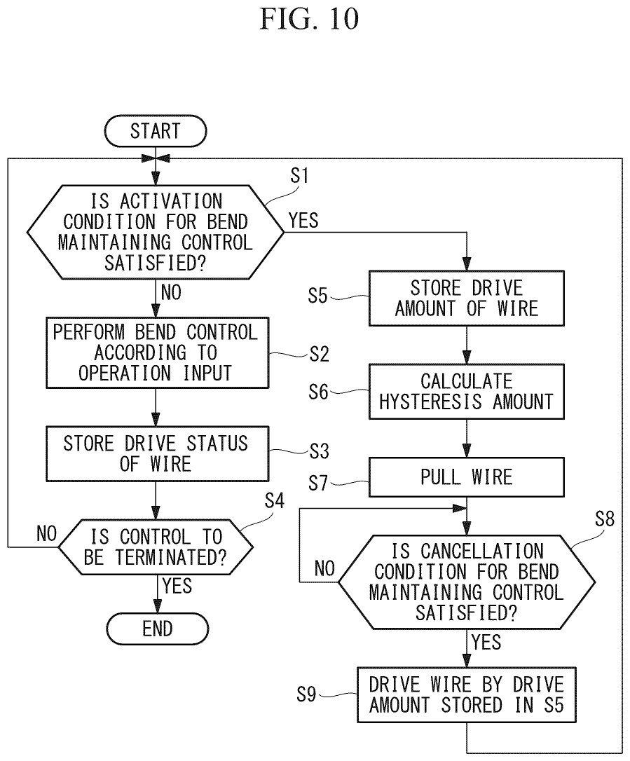

The endoscope control method executed by the endoscope control device 40 according to this embodiment will be described in further detail with reference to a flowchart in FIG. 10.

First, when the control of the endoscope 10 commences, it is determined whether or not an activation condition for the bend maintaining control is satisfied (step S1).

The activation condition for the bend maintaining control is, for example, any of C1 to C3 below.

C1: A predetermined time period lapses after the bend angle of the bending section 21 changes from the first angle θ1 to the second angle θ2.

C2: An input for requesting activation of the bend maintaining control is received from the operator, such as a surgeon.

C3: The treatment-tool sensor 35e detects that the treatment tool 80 is inserted into the channel tube 17.

The activation condition C1 is satisfied in a case where, for example, a timer (not shown) included in the endoscope control device 40 starts a timekeeping process and determines that a predetermined time period has lapsed when an operation input for decreasing the bend angle from the first angle θ1 to the second angle θ2 is received.

The activation condition C2 is satisfied when the switch 55 provided in, for example, the manipulation device 50 is switched or when the audio sensor 56 provided in, for example, the manipulation device 50 detects the voice of the operator requesting activation of the bend maintaining control.

The activation condition C3 is satisfied when the treatment-tool sensor 35e provided in the coupling section 12 detects the treatment tool 80 extending through the channel tube 17. For example, if the channel tube 17 is used for suctioning a drain fluid from inside the body, the activation condition C3 for the bend maintaining control may be set so as to be not satisfied when a suction button (not shown) is being pressed, thereby preventing erroneous detection between the drain fluid and the treatment tool 80.

If none of the activation conditions C1 to C3 is satisfied in step S1, the processor 41 of the endoscope control device 40 executes normal bending control of the bending section 21 by the driving device 30 in accordance with the operation input received by the manipulation device 50 (step S2). Then, the processor 41 stores the drive status of the wire 16 into the memory 42 (step S3).

The drive status of the wire 16 is data for obtaining the relationship among the drive amount of the wire 16, the tension of the wire 16, and the bend angle shown in FIG. 8. An example includes, in a case where the endoscope 10 includes a shape sensor (not shown) for detecting the shape thereof, a data pair of the bend angle determined from the shape detected by the shape sensor and the drive amount of the wire 16 determined from the rotational amount of the motor 34 detected by the encoder 34a. The shape sensor used may be an arbitrary sensor, such as a magnetic sensor (UPD) or an optical fiber sensor (FBG). Alternatively, the drive status of the wire 16 may be a data pair of the tension detected by the tension sensor 35a and the drive amount of the wire 16 determined from the rotational amount of the motor 34 detected by the encoder 34a.

Subsequently, it is determined whether or not the control is to be terminated (step S4). If the control is not to be terminated, the process from step S1 is repeated. By repeating the process from step S1 to step S4 without terminating the control, multiple data pairs of the rotational amount of the motor 34 and the bend angle or the tension of the wire 16 are stored. Accordingly, at least a part of the relationship among the drive amount of the wire 16, the tension of the wire 16, and the bend angle shown in FIG. 8 can be estimated. The hysteresis curve shown in FIG. 8 may be estimated by detecting the start of bending when the bend angle is to be returned to the second angle θ2 from an endoscopic image or the rate of change in tension and performing linear interpolation.

If any of the activation conditions C1 to C3 is satisfied in step S1, the processor 41 stores the drive amount of the wire 16 at that point into the memory 42 (step S5). Then, a so-called dead zone is calculated based on the drive status of the wire 16 stored in step S3 and the current drive amount of the wire 16 (step S6), and the wire 16 is pulled by a drive amount smaller than the dead zone (step S7).

For example, a dead zone is a drive-amount difference AD to an intersection point between a straight line extending parallel to the abscissa axis from the second angle θ2 on a hysteresis curve of the bend angle relative to the drive amount of the wire 16 in FIG. 8 and a hysteresis curve when the tension of the wire 16 is increased along the arrow Y1. When the drive amount of the wire 16 is smaller than the difference ΔD, the tension of the wire 16 can be increased without causing the bend angle to change. However, the driving can be performed by a drive amount similar to the difference ΔD as much as possible without exceeding the difference ΔD.

Alternatively, instead of increasing the drive amount by the drive-amount difference ΔD, the tension of the wire 16 may be increased to a tension not exceeding a third tension T3 determined in accordance with a first tension T1 when the bending is performed to the first angle θ1 by pulling the wire 16 and a second tension T2 when the bend angle is returned to the second angle θ2 by loosening the wire 16.

Subsequently, it is determined whether or not a cancellation condition for the bend maintaining control is satisfied (step S8).

The cancellation condition for the bend maintaining control is, for example, any of C4 to C6 below.

C4: An operation input for instructing a new bending operation is received during the bend maintaining control.

C5: An input for requesting cancellation of the bend maintaining control is received from the operator, such as a surgeon.

C6: The treatment-tool sensor 35e detects that the treatment tool 80 is removed from the channel tube 17.

When any of the cancellation conditions C4 to C6 is satisfied, the wire 16 is loosened to the drive amount of the wire 16 stored in step S5 (step S9), and the process from step S1 is repeated. In the case of the cancellation condition C4, the switch 55 used in the activation condition C2 needs to be a momentary switch.

Accordingly, with the medical system 100, the endoscope control device 40, and the endoscope control method according to this embodiment, the bend maintaining control is executed by the processor 41 when the bend angle is decreased by loosening the wire 16. Although the tension of the wire 16 decreases rapidly when the bend angle is decreased, the tension of the wire 16 can be increased without causing the bend angle to change by executing the bend maintaining control.

As a result, when the treatment tool 80 is inserted into the channel tube 17 in a state where the bend angle is decreased, a change in the bend angle caused by the rigidity of the treatment tool 80 can be prevented. Specifically, when the treatment tool 80, which is highly rigid, is inserted into the channel tube 17, the bending section 21 receives a force in the direction that decreases the bend angle from the treatment tool 80. However, the tension of the wire 16 is preliminarily increased in accordance with the bend maintaining control, so that the bend angle does not have to be changed.

Accordingly, this is advantageous in that, for example, after the second angle θ2 is reached from the first angle θ1, a change in the bend angle, that is, a change in the visual field of the endoscope 10, can be prevented even if the treatment tool 80 is inserted into the channel tube 17. The operator, such as a surgeon, can readily perform a treatment using the treatment tool 80 in accordance with the non-changing visual field.

In this embodiment, when any of the cancellation conditions C4 to C6 is satisfied in step S8, the wire 16 is loosened to the drive amount of the wire 16 stored at the start of the bend maintaining control in step S9. Accordingly, the operational state by the operator at the start of the bend maintaining control is recovered, so that the operator can operate the endoscope with a sense of operation similar to when the bend maintaining control is not performed.

If the hysteresis (dead zone) of the bending operation is large, compensation control involving driving the wire 16 at high speed is performed by estimating the dead zone. In this case, if the tension of the wire 16 remains to be increased in accordance with the bend maintaining control, an estimation error of the dead zone increases. By loosening the wire 16 to the state at the start of the bend maintaining control when the bend maintaining control is canceled, the wire 16 can be driven with high responsiveness without being affected by the estimation error of the dead zone in the compensation control.

Although the tension sensor 35a that detects the tension of the wire 16 is described as an example of the first sensor in this embodiment, the configuration is not limited to this. The first sensor employed may be the torque sensor 35b that acquires a drive torque as information related to a change in the tension of the wire 16 or an electric current sensor that acquires an electric current value of the motor 34. The acquired drive torque or the acquired electric current value may be converted into the tension of the wire 16.

### REFERENCE SIGNS LIST

10 endoscope
16 wire
21 bending section
30 driving device
40 endoscope control device
41 processor
42 memory
43 storage unit (storage medium)
44 input-output control unit (interface)
80 treatment tool
100 medical system
θ1 first angle
θ2 second angle

1. An endoscope control device comprising:
at least one processor comprising hardware, the at least one processor being configured to:
control an actuator connected to at least one wire of an endoscope to drive the at least one wire and bend a bending section at a distal end of the endoscope to a first angle, and
when a bend angle of the bending section is returned to a second angle smaller than the first angle by loosening the at least one wire from the state where the bending section is bent to the first angle, control the actuator to drive the at least one wire to maintain the bend angle at the second angle.

2. The endoscope control device according to claim 1, wherein the at least one processor is configured to maintain the bend angle at the second angle based on a change in tension of the at least one wire acquired when the bending section is bent from the first angle to the second angle.

3. The endoscope control device according to claim 1, wherein the at least one processor is further configured to:
receive a bending operation instruction input by an operator, and
cancel the maintaining of the bend angle at the second angle when the bending operation instruction is received.

4. The endoscope control device according to claim 1, wherein the at least one processor is configured to:
execute the maintaining of the bend angle at the second angle in accordance with insertion of a treatment tool into a channel of the endoscope, and
cancel the maintaining of the bend angle at the second angle in accordance with removal of the treatment tool from the channel.

5. The endoscope control device according to claim 1, wherein the at least one processor is further configured to:
access a stored initial drive amount of the at least one wire when maintaining the bend angle at the second angle is started, and
when cancelling the maintaining of the bend angle at the second angle, control the actuator to loosen the at least one wire to the stored initial drive amount.

6. An endoscope control method comprising:
controlling an actuator connected to at least one wire of an endoscope to drive the at least one wire and bend a bending section at a distal end of the endoscope to a first angle, and
when a bend angle of the bending section is returned to a second angle smaller than a first angle by loosening the at least one wire from a state where the bending section is bent to the first angle, using at least one processor to control the actuator to drive the at least one wire to maintain the bend angle at the second angle.

7. The endoscope control method according to claim 6, wherein the maintaining of the bend angle at the second angle is based on a change in tension of the at least one wire acquired when the bending section is bent from the first angle to the second angle.

8. The endoscope control method according to claim 6, further comprising:
receiving a bending operation instruction input by an operator, and
cancelling the maintaining of the bend angle at the second angle when the bending operation instruction is received.

9. The endoscope control method according to claim 6, wherein method further comprising:
executing the maintaining of the bend angle at the second angle is in accordance with insertion of a treatment tool into a channel of the endoscope, and
cancelling the maintaining of the bend angle at the second angle is in accordance with removal of the treatment tool from the channel.

10. The endoscope control method according to claim 6, further comprising:
accessing a stored initial drive amount of the at least one wire when the maintaining of the bend angle at the second angle is started, and
when cancelling the maintaining of the bend angle at the second angle, control the actuator to loosen the at least one wire to the stored initial drive amount.

11. A computer-readable non-transitory storage medium that stores an endoscope control program causing a computer to execute the endoscope control method according to claim 6.

12. A medical system comprising:
an endoscope comprising an insertion section having a bending section and at least one wire connected to the bending section, the bending section being disposed at a distal end of the insertion section;
an actuator connected to the at least one wire and configured to drive the at least one wire and bend the bending section; and
the endoscope control device according to claim 1.

13. The medical system according to claim 12, further comprising a sensor that is configured to detect a change in tension of the at least one wire,
wherein the processor is configured to maintain the bend angle at the second angle based on the detected change in tension of the at least one wire when the bending section is bent from the first angle to the second angle.

14. The medical system according to claim 13, wherein the first sensor is a tension sensor disposed at a proximal end of the at least one wire and is configured to detect the tension of the at least one wire.

15. The medical system according to claim 12, further comprising an interface to which a bending operation instruction by an operator is input,
wherein, subsequent to maintaining the bend angle at the second angle, the processor is configured to cancel maintaining the bend angle at the second angle when the bending operation instruction is received.

16. The medical system according to claim 12, wherein
the endoscope comprises a channel into which a treatment tool is insertable,
the medical system further comprises a sensor configured to detect whether the treatment tool is inserted into or removed from the channel, and
the processor is configured to:
execute the maintaining of the bend angle at the second angle when the sensor detects that the treatment tool is inserted, and
cancel the maintaining of the bend angle at the second angle when the sensor detects that the treatment tool is removed.

17. The medical system according to claim 16, wherein the sensor is disposed a position along a longitudinal length of the channel.

18. The medical system according to claim 12, wherein
the endoscope control device comprises at least one memory,
the memory stores an initial drive amount of the at least one wire when the maintaining of the bend angle at the second angle is started, and
when cancelling the maintaining of the bend angle at the second angle, the processor is configured to control the actuator to loosen the at least one wire to the stored initial drive amount.
